(19) United States
(12) Patent Application Publication
(10) Pub. No.: US 2025/0111888 A1
(43) Pub. Date: Apr. 3, 2025

(54) Systems And Methods For Enhancing Crop Simulations

(71) Applicant: MONSANTO TECHNOLOGY LLC, Saint Louis, MO (US)

(72) Inventors: Neal CARPENTER, Wildwood, MO (US); Megan GILLESPIE, Wildwood, MO (US); Wei-Ting LIAO, Ballwin, MO (US); Chenyong MIAO, Saint Louis, MO (US)

(21) Appl. No.: 18/898,001

(22) Filed: Sep. 26, 2024

Related U.S. Application Data
(60) Provisional application No. 63/541,771, filed on Sep. 29, 2023.

(51) Int. Cl. G16B 5/00; G16B 20/00

(52) U.S. Cl. CPC G16B 5/00; G16B 20/00

(57) ABSTRACT

Systems and methods are provided for simulating one of more traits associated with seed products in multiple fields. One example computer-implemented method includes retrieving, by a simulation computing device, from a data structure, data specific to multiple seed products and multiple fields, where the data includes a first mode of data and a second mode of data, and simulating a trait of interest for the multiple seed products and/or multiple fields, based on a simulation architecture. The simulation architecture includes a first model specific to the first mode of data, a second model specific to the second mode of data, and a neural network coupled to the output of the first and second models as an aggregation layer. The computer-implemented method also includes transmitting the simulated trait of interest for the multiple seed products and/or multiple fields to a grower and/or an agricultural device associated with the multiple fields.

### CROSS-REFERENCE TO RELATED APPLICATION

This application claims the benefit of, and priority to, U.S. Provisional Application No. 63/541,771, filed on Sep. 29, 2023. The entire disclosure of the above-referenced application is incorporated herein by reference.

### FIELD

The present disclosure generally relates to systems and methods for simulating crops and to enhancing such crop simulations, and in particular, to systems and methods related to simulating traits of crops, such as, for example, ear height, as an indication of suitability of the crops for fields, and enhancements relating thereto.

### BACKGROUND

This section provides background information related to the present disclosure which is not necessarily prior art.

Crops are often selected for fields based on a preference of growers associated with the fields and/or suitability of the crops to/for the fields. For example, a seed product may be selected for a field based on the expected yield of the seed product and/or field, or the suitability of the seed product to the specific climate of the field. The suitability of the seed product to the field, then, is typically based on historical performance of the seed product in the same or other fields, if available.

### DRAWINGS

The drawings described herein are for illustrative purposes only of selected embodiments and not all possible implementations, and are not intended to limit the scope of the present disclosure.

FIG. 1 illustrates an example system of the present disclosure for simulating traits of seed products for specific fields, whereby ones of the seed products are selected to be planted in the fields based on the simulated traits;

FIG. 2 is a block diagram of an example computing device that may be used in the system of FIG. 1;

FIG. 3 is an example method, suitable for use with the system of FIG. 1, for simulating traits of seeds products for planting in one or more specific fields and for selecting seed products for the field(s) based on the simulation; and

FIGS. 4A-4B illustrate example graphs indicative of performance of a simulation architecture included in the system of FIG. 1, based on the example method of FIG. 3, in connection with simulating traits of seeds products for planting in one or more specific fields.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Suitability of a seed product for a field is difficult to determine. For particular performance traits, such as, for example, ear height, etc., it is important to accurately predict or simulate the crop performance as to that trait. Ear height, for example, may be inconsistent with harvesting equipment (e.g., combines, etc.) when the ears of corn are too low (e.g., below about twenty inches, about twenty-four inches, etc.). As such, where a plant grown from a seed product does not reach the minimal ear height for proper harvest, based on the seed product performance in the specific environment, with its specific genetics, the resulting harvest is diminished. Accordingly, accurate simulation of particular traits, based on limited data, is a technical problem which directly impacts selection of seed products for specific fields and associated environments, and further impacts overall performance of the growing operations in those fields.

Uniquely, the systems and methods herein provide for simulating traits associated with seed products in multiple fields based on data representative of the seed products and environments of the multiple fields.

In particular, a simulation architecture is employed, which relies on genetic data of the seed products, environmental data for the fields (e.g., weather data, soil data, etc.), and/or management practices for the fields. The simulation architecture includes a combination of models to convert the data to latent features associated with a trait of interest, which are combined through an aggregation model to simulate the desired trait(s) of the seed products in the specific fields. In this manner, an accurate simulation of the seed products, for the specific trait(s), is defined, whereby decisions related to planting of the seed products in the fields based on the specific trait(s) are achieved. For example, certain short corn seed products may be designated suitable or not for a field based on the simulated ear height trait indicating the crop will be suitable (or not) for harvest in the field by conventional combines (e.g., the simulated ear height trait indicates an ear eight above a minimum threshold, etc.).

As such, the simulation architecture of the present disclosure provides a technical solution to the technical problem of accurately assessing traits in seed products to be grown in certain fields, which impacts the suitability of (and, potentially, the selection of) the seed products for the certain fields.

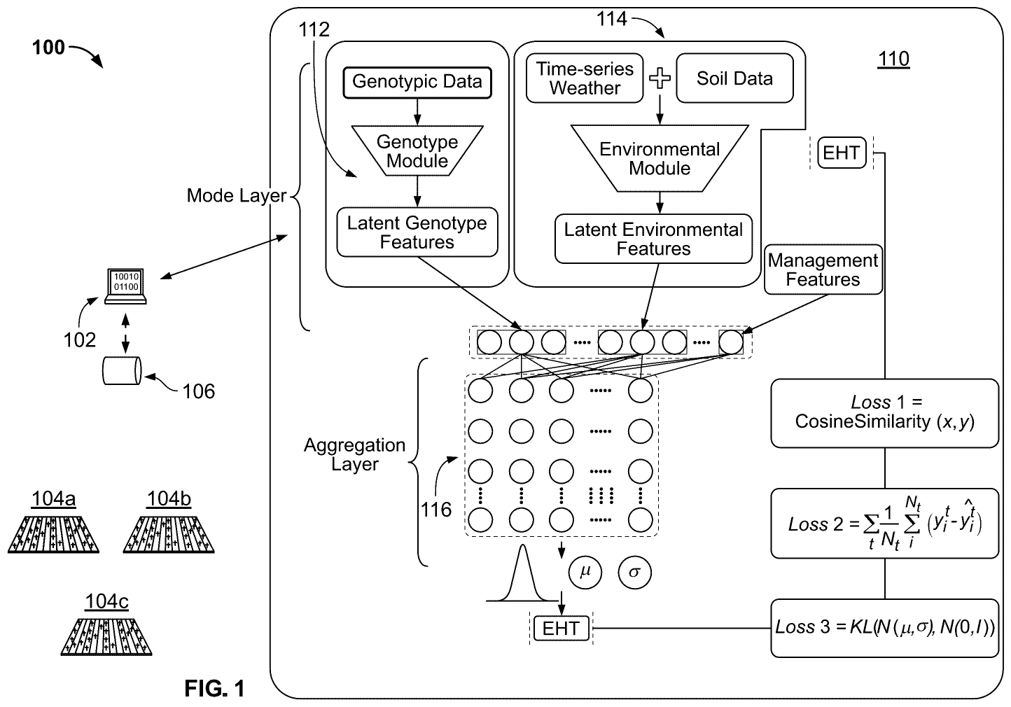

FIG. 1 illustrates an example system 100 for simulating traits of seed products to be planted in multiple fields, based on data associated with the fields and/or the seed products. In the example embodiment herein, for purposes of illustration, the simulation is described with reference to ear height of a corn plant as the trait (e.g., the trait of interest, the desired trait, etc.). That is, short corn may have advantages related to sustainability, standability, etc., but may be precisely assigned to specific fields in specific environments to account for at least harvest conditions (e.g., limitations on combines for harvesting, etc.) as explained herein. That said, depending on products to be planted, environmental conditions, goals, objectives, etc., it should be appreciated, as indicated further below, that the simulated trait may be otherwise in other example embodiments (e.g., other than ear height for corn, etc.).

While the system 100 is described herein with reference to simulating corn ear height, it should be appreciated that the system 100 may also be used for simulating other traits within the scope of the present disclosure. For instance, the system 100 may be used for simulating, without limitation, traits relating to size and/or heartiness of plants (e.g., plant height, standability, sustainability, stalk girth, stalk strength, etc.), yield, time to maturity, traits relating to resistance to stress(es) (e.g., disease or pest resistance, etc.), traits relating to resistance to abiotic stress(es) (e.g., drought or salinity resistance, etc.), growing climate, thousand kernel weight, saleable seed units, average seed pixel area, tassel size and skeletonization, grey leaf spot, anthracnose, goss's wilt, diplodia, fusarium, gibberella, northern leaf blight, brown/southern rust, tar spot, greensnap, moisture, lodging, chloride, southern stem canker, white mold, sudden death syndrome, soybean cyst nematode, root knot nematode, phytophthora, iron deficiency chlorosis, frogeye leaf spot, brown steam rot, maturity, any other suitable phenotypic trait, and/or combinations thereof.

As shown, the system 100 includes several fields, which are referenced 104a-c. It should be understood that the three fields 104a-c are illustrative of many fields, including tens, hundreds, thousands of fields, etc. In general, a field is an area of land, which may range from less than an acre to a hundred acres, or several hundreds or thousands of acres, or sizes therebetween. The field 104 is defined at a specific geographical location, and may be described by a boundary of the field relative to the location. In connection with the description herein, the fields 104a-c may include all fields associated with a particular grower, or a subset of the fields associated with a grower. In addition, in some examples, the fields 104a-c may include an enclosed growing space such as a greenhouse, etc.

The fields 104a-c may be planted, presently or in prior growing seasons, with various different crops and/or different varieties of the same crops, etc. For instance, the fields 104a-c may be populated with one or more different crops (or plants) such as (without limitation) corn (or maize), wheat, beans (e.g., soybeans, etc.), peppers, tomatoes, tobacco, eggplant, corn or maize, rice, rye, sorghum, sunflower, potatoes, cotton, sweet potato, coffee, coconut, pineapple, citrus trees, prunes, cocoa, banana, avocado, fig, guava, mango, olive, papaya, cashew, almond, sugar beets, sugarcane, oats, barley, vegetables, or other suitable crops or products or combinations thereof, etc. Or, the fields 104a-c may each include the same type of plants/crops, or a number of different varieties of the same type of plants (or crops). For example, field 104a may include a first hybrid maize plant, while fields 104b-c may include a second, different hybrid maize plant.

As shown in FIG. 1, the system 100 also includes a simulation computing device 102 which is associated with a database 106. The database 106 is configured to store data related to the crops and/or the fields 104a-c.

In particular, the crops in the fields 104a-c, or crops desired to be planted in the fields 104a-c, may be consistent with any of the types of crops listed above. In this example embodiment, the type of crop is corn, or maize. The seed products, then (from which the crops are grown), may include various different varieties of corn. For the different varieties of corn (e.g., dozens, hundreds, thousands, etc., of varieties), the database 106 includes genotypic data representative of the specific seed products (e.g., based on historical data accumulated and stored in the database 106 for the different varieties of corn (and other seed products), etc.). That is, each different variety of corn is associated with a unique nucleotide sequence (e.g., where the parental line thereof may have 5123 markers, etc.). The genotypic data, then, is an expression of that sequence as a vector of values, where each value represents a marker in the nucleotide sequence. In this example, the vector includes thousands of markers for the male and female contributors of a seed product, where the location in the vector is indicative of the particular marker, and the value in the vector (e.g., −1, 0, 1, etc.) then indicates the content of the marker for that particular nucleotide sequence of the variety. With regard to the values in the vector, a value of −1 and 1 may indicate homozygous genotypes and a value of 0 may indicate heterozygous genotypes. In addition, the genotype data for the hybrid product may further be represented by (or together with) a matrix with dimensions of (5123, 2), where 5123 is the number of markers from each parent and 2 is the number of parental lines.

In this way, the genotypic data for the variety is sufficient to reproduce, to some degree, the nucleotide sequence of the variety represented by the vector. The one or more thousands of markers (or more or less) may be selected, in various embodiments, based on distinctions between the nucleotide sequences for the different varieties, whereby the genotypic data is more directed to the uniqueness of the seed product (rather than the commonalities).

It should be appreciated that the genotypic data may be represented in a different form in other embodiments, whereby the genotypic data is representative of the genetic uniqueness of the different varieties and/or suitable to be ingested to simulate one or more traits, as explained in more detail below.

In addition to the genotypic data, the database 106 also includes weather data for the fields 104a-c. The weather data is associated with identifying data for one or more of the fields 104a-c (e.g., via field identifier, etc.), for example, to identify the specific weather data, over time, to the specific field(s) a-c. In doing so, the weather data may include a daily resolution in the illustrated embodiment (however, it may include other resolutions in other example embodiments (e.g., hourly, bi-daily, weekly, etc.)).

The weather data, for example, may include, without limitation, atmospheric pressure (e.g., average, minimum, maximum, etc.), wind (e.g., maximum gust, minimum speed, maximum speed, average speed, direction, etc.), precipitation (e.g., rate, maximum rate, average rate, volume, etc.), solar radiation (e.g., daylight duration, maximum net radiation, etc.), cloud cover (e.g., average, etc.), snow (e.g., cover, depth, density, etc.), soil temperature at different levels (e.g., levels 1-4, etc.) (e.g., average, minimum, maximum, etc.), temperature (e.g., minimum, maximum per interval (e.g., day, hour, etc.), etc.), dew point temperature (e.g., average, minimum, maximum, etc.), relative humidity (e.g., average, minimum, maximum, etc.), etc. The weather data may be collected from planting to harvest, for example, in each of the fields 104a-c (and/or regions including the fields, etc.). In addition, as different weather features have different units resulting in different ranges of each feature, the weather data may be normalized in example embodiments, for instance, using a mean and standard deviation, etc. to provide values of all weather features are in a generally similar range.

In this example embodiment, the weather data may be expressed in a time-series over a regular or irregular interval. For example, the average daily temperature, along with the maximum temperature and daylight duration, may be compiled in a time series data structure for each of the fields 104 (and stored in the database 106).

Further, the database 106 may include soil data. The soil data is associated with identifying data for the corresponding fields 104a-c (e.g., via field identifier, etc.), for example, to identify the specific soil data, over time, to the specific field(s). The soil data, for example, may include, without limitation, measurements relating to organic matter (OM), National Commodity Crop Productivity Index (NCCPI), cation exchange capacity (cec), ph (e.g., acidity, etc.), sand content (or percentage), clay content (or percentage), silt content (or percentage), available soil water capacity (volumetric fraction until) wilting point (wwp), and bulk density, etc., which may be captured at one of more discrete times, or intervals, prior to, during, or after the growing of the plants/crops in the fields 104a-c.

It should be appreciated that the weather data and/or soil data may be referred to herein as environmental data for the fields 104.

It should be appreciated that in one or more one example embodiments, the simulation computing device 102 may be configured to pre-process the weather data and the soil data and store the same in the database 104 for input to simulation architecture 110. For example, and as discussed above, the weather data may include a max temperature per hour, an average pressure per day, and a maximum net solar radiation per day, etc., which are expressed daily, from planting until harvest of the historical crop/fields. The weather data, for example, may be derived or received from different sources, such as, for example, the Copernicus Climate Change Service (C3S) (e.g., ERA5 data (e.g., in 27 km resolution, etc.), etc.), TWC sources (e.g., History on Demand (HoD data, etc.) (e.g., in 4 km resolution, etc.), TWC high-resolution precipitation (e.g., in 8 km resolution, etc.), etc.), etc. To put the different data sources together, the computing device 102 may be configured to first map the data to a specific field based on the field geometry. In doing so, the computing device 102 is aware of which data source grids (e.g., ERA5, TWC HoD, and TWC high-resolution precipitation, etc.) are associated with the field. Then, based on grid IDs for the grids, the computing device 102 is configured to retrieve the corresponding weather data from the database 106. The retrieved data may include different units, different resolutions, etc. Therefore, the simulation computing device 102 may further be configured to normalize and/or resize the weather data to account for differences in units, numerical scales, resolutions, etc., to provide a weather data structure (e.g., in the same or similar scale, etc.) (broadly, an environmental data structure). For instance, the weather data may be normalized using a mean and standard deviation, etc. to provide values of all weather features are in a generally similar range. That said, it should be appreciated that the data may be normalized in other manners within the scope of the present disclosure. The soil data may also be normalized or resized as necessary in certain embodiments as well. The weather data structure then includes, for example, data spanning 180 days from planting to harvest and N weather features (e.g., max temperature, average pressure, etc.) to which the soil data is appended, i.e., M soil features (e.g., OM, NCCPI, etc.). The resulting weather data structure is 180 by (N+M) in this example.

It should be appreciated that the specific form, format and size of the weather data structure may be specific to the trait of interest, the simulation architecture, etc. Or, it may be general to the seed products, etc.

While the above description of the database 106 is limited to genotypic data, weather data and soil data, it should be appreciated that other types of data may be included in the database 106 for the crops and/or the fields 104a-c, or region(s) in which the fields 104a-c are located, etc. For example, the database 106 may include image data for the fields 104a-c, prior to, during and/or after planting or harvesting (or any time there between) of the fields 104a-c, in association with identifying data for one or more of the fields 104a-c (e.g., via field identifier, etc.) and/or crops in the fields 104a-c (e.g., via plant identifiers, etc.), for example, to identify the specific image data, over time, to the specific field(s)/plant(s), etc. The image data may be included, for example, in simulations of trait(s) related to physiological and/or observable traits of the crop(s). The image data may include red (R), green (G), blue (B), near-field infrared (NIR) data, or any other suitable band(s) of data, or any derivation thereof (e.g., NDVI or other vegetative indexes, etc.). In this regard, sensors may be employed either in or near the fields 104a-c, for example, as unmanned aerial vehicles (UAV) (e.g., drones, etc.), ground-based vehicles, or apart from the fields 104a-c, as, for example, satellites, etc.

In addition to images, other types of data (included in the database 106) may further include management data for the fields 104a-c. Management data may include any indication of one or more management practices, such as, for example, tillage, rotation, planting, treatments, irrigation, etc., for the fields 104a-c, prior to, during or after the growing of the plants/crops in the fields 104a-c. The data, again, may be received, retrieved, generated, etc. and included in the database 106 and associated with identifying data for one or more of the fields 104a-c and/or crops included in the fields 104 (e.g., via field identifier, plant identifier, etc.), for example, to identify the specific management practice data, over time, to the specific field(s) 104a-c.

Further, other types of data may include data generated by, provided by, received from, etc. modalities such as novel sensors, etc. (e.g., multispectral data, data from aerial sensors and/or under canopy sensors, satellite data/information, in-field disease detection data, data from root sensors (e.g., sonar data, x-ray data, etc.), etc.).

It should be understood that the database 106 includes the above data for a number of years, where applicable. As such, for weather data, for example, the database 104 includes multiple years of weather data, in raw data for and/or in time series data structures, for specific fields, region, etc.

Beyond the above data, the database 106 includes historical phenotypic data for crops grown in the fields 104a-c over the past years. The phenotypic data may include, for example, any trait of interest associated with the crop(s) grown in the fields 104a-c (or in other fields, etc.). For example, the phenotypic data may include ear height for corn, yield, moisture content (e.g., over time, at harvest, etc.), plant height, or test weight, etc. The historical phenotypic data includes an identification of the particular variety (ies) of crop(s) included in the fields 104a-c, which may be linked to the genotypic data described above, and field identifiers (e.g., field numbers, boundary, locations, etc.), timing, etc., which may be linked to the weather data, soil data, and other data described above. In this manner, the database 106 includes a training set of data (e.g., genotypic data, environmental data, other data and trait data, etc.), which may be used, as described below.

It should be further appreciated that the phenotypic data may be expressed in a manner consistent with the data in the database 106. For example, where the field 104a includes two short corn hybrids, yet the weather data is defined at the field level, the phenotypic data may be expressed as an average or mean (e.g., mean ear height, etc.) for the field 104a for both of the hybrids to avoid data leakage in the training data set based on limitations of the data, such as, for example, the weather data, etc.

With continued reference to FIG. 1, in this example embodiment, the simulation computing device 102 is configured by the simulation architecture 110 to operate as described herein. The simulation architecture 110 configures the simulation computing device 102 to simulate a trait (e.g., ear height, yield, other traits described herein, etc.) of a seed product in one or more of the fields 104a-c.

As indicated above, the trait in this example is ear height for corn. Specifically, in one or more instances, short corn, which is designed to grow in a particular manner, is used in fields. However, the corn may not reach a minimum height due to unfavorable combinations of genetics, environment and management practices. In such instances, the corn grown in the field(s) 104a-c is troublesome to harvest, or yield is reduced for ears of corn that do not attain the minimum height for proper harvesting (e.g., based on combine specifications, etc.). To limit the potential for such instances, the simulation architecture 110 configures the simulation computing device 102 to simulate the ear height of short corn, prior to planting, based on the conditions of the fields 104a-c, etc., to provide confidence that a particular variety of short corn is compatible with the specific fields 104a-c, environmental conditions of the fields 104a-c, management practice(s) for the fields 104a-c, etc., while still providing growth performance to conform to harvest criteria for the grower (e.g., based on specific combines or other pickers used by the grower to harvest the fields 104a-c, etc.).

As shown, the simulation architecture 110 includes a mode layer, in which multiple modes of data (as described above) are input (individually or together) and an aggregation layer, in which the different modes of data are combined to provide a simulation of the trait of interest. The example mode layer includes multiple separate modes of data (e.g., soil, weather, genotypic, soil+weather, weather+genotypic, management, etc.) and which configures the simulation computing device 102, for each mode of data, to convert the input data into latent data. The aggregation layer, in turn, configures the simulation computing device 102 to receive the latent feature data from the mode layer as input in one or more forms (e.g., via feature fusion (e.g., a concatenation of the latent data features, etc.), etc.), including or based on the trait of interest, etc., and to combine the latent variables to simulate an output indicative of the trait of interest based on the combined modes of latent data.

It should be appreciated that the multi-modal aspect of the simulation architecture 110 is innovative, important and/or critical, in this example embodiment. By permitting multiple different modes of data to be processed through different models in the mode layer, before aggregation in the aggregation layer, the different models of the model layer are configurable, tailorable and/or selectable to the specific features and/or profile(s) of the data for that specific mode. In this way, in this example embodiment, the disparate data types (e.g., genetic data, weather data, and soil data, etc.) are assessed in the individual models, as explained below, and combined, whereby the impact of one type of data is not limited or otherwise diminished by the other data (e.g., in that particular models are utilized for the particular different data, etc.), etc.

In more detail in the example embodiment of FIG. 1, the simulation architecture 110 includes a genotype mode 112 (including a genotype model or module) and an environmental mode 114 (including an environmental model or module) in the mode layer, each of which have deep learning models (e.g., the genotype model and the environmental model, etc.). The genotype mode 112 may include, for example, a bi-directional long short-term memory (LSTM) model, which is configured to input marker vectors from parental lines to generate latent genotypic feature data; and the environmental mode 114 may include a LSTM/RNN (long short-term memory/recurrent neural network) model, which is configured to input a combination of weather data and soil data to generate latent environmental features. More generally, each of the modes 112, 114 includes a deep learning model. Further, the latent genotypic feature data, latent environmental feature data, and management practice data are combined (e.g., expressed as a concatenation of the latent features and trait of interest, etc.) in feature combination (or feature fusion) between the mode layer and the aggregation layer. That said, it should be appreciated that the genotype model may include one or more of a deep neural network (DNN) model, a convolutional neural network (CNN) model, a recurrent neural network (RNN) model, a generative pre-trained (GPT) model, a multilayer perceptron (MLP) model, and/or a long short-term memory (LSTM) model. The environmental model may include one or more of a DNN model, CNN model, a RNN model, a GPT model, a MLP model, and/or a LSTM model.

In this example embodiment, the specific parameters of the LSTM model for the genotype mode 112 are provided in Table 1 (e.g., Table 1 illustrates the model architecture for genotype data, etc.), and the specific parameters of the LSTM/RNN model for the environmental mode 114 ate provided in Table 2. In connection therewith, the model architecture for the genotype data of Table 1 is composed of multiple convolutional layers, max pooling layers and dense layers. The multiple layers are designed to capture genotypic features from the input genotype data. Similarly, in Table 2, multiple layers are designed to capture weather features from time-series weather data.

It should be appreciated that, despite the specific example below, other architecture embodiments may be included in other system embodiments, while changes to the architecture (e.g., more or less layers, filters, kernel sizes, pool sizes, etc.) may be included in still other embodiments. In other words, the architectures illustrated in Tables 1 and 2 below are example in nature, and other architectures may be used herein with in the scope of the present disclosure.

TABLE 1

| Layer | Output Dimension | Filters | Kernel Size | Pool_Size/Stride |
|---|---|---|---|---|
| InputLayer | (5123, 8) | 32 | 5 | 2 |
| Conv1D | (2560, 32) | | | |
| Conv1D | (1278, 32) | 32 | 5 | 2 |
| MaxPooling1D | (426, 32) | 48 | 5 | 3 |
| Conv1D | (422, 48) | | | 1 |
| Conv1D | (418, 48) | 48 | 5 | 1 |
| MaxPooling1D | (209, 48) | | | 2 |
| Conv1D | (205, 48) | 48 | 5 | 1 |
| Flatten | (9840, ) | | | |
| Dropout (0.3) | (9840, ) | | | |
| Dense | (128, ) | | | |
| Dense | (64, ) | | | |

TABLE 2

| Layer | Output Dimension | Filters | Kernel Size | Pool_Size/Stride |
|---|---|---|---|---|
| InputLayer | (180, 51) | 48 | 7 | 1 |
| Conv1D | (175, 48) | | | |
| Conv1D | (169, 64) | 64 | 7 | 1 |
| MaxPool1D | (84, 64) | 128 | 5 | 2 |
| Conv1D | (80, 128) | | | 1 |
| Conv1D | (76, 128) | 128 | 5 | 1 |
| MaxPool1D | (38, 128) | | | 2 |
| Conv1D | (36, 128) | 128 | 3 | 1 |
| Flatten | (4608, ) | | | |
| Dropout (0.3) | (4608, ) | | | |
| Dense | (64, ) | | | |

The aggregation layer, then, in this example, includes a feed-forward neural network 116, which is configured to generate a trait of interest output by combining the latent feature data from the mode layer, via the feature data combination. The feed-forward neural network 116 includes several nodes (e.g., hundreds, thousands, million of nodes, etc.), which are trained, as explained below, to produce a trait from input data, which in this example, is the latent data from the mode layer. It should be understood that other types of neural networks, or other models, may be included in the aggregation layer in other simulation architecture embodiments, and be otherwise consistent with FIG. 1.

The simulation architecture 110 is suited for simulating corn ear height in this example embodiment. In such an embodiment, as explained above, the training data set may include data from over one hundred fields (e.g., including the fields 104a-c, etc.) and including over ten thousand different corn varieties, and may include many different weather features (e.g., as explained above, etc.), multiple soil features and thousands of markers from the parental lines of the corn varieties, etc. In connection therewith, the simulation computing device 102 is configured to access a training data set from the database 106 and to train the simulation architecture 110 to simulate corn ear height based on the genotypic data, environmental data and management practices included therein.

As indicated in FIG. 1, the time series weather data and the soil data are joined prior to input to the environment mode 114 of the simulation architecture 110. It should be appreciated that the same or other input data and/or specific representations/combinations of that data, per mode, may be used in other implementations of the embodiment in FIG. 1, and in still other embodiments other than (or beyond) FIG. 1. For example, the weather data and the soil data may be separated into different mode models, whereby the mode layer would include three different mode models (or modules).

In connection with training the simulation architecture 110 (e.g., as an ear height simulator, etc.), the simulation computing device 102 may consider, evaluate, etc. different hyperparameters (and/or combinations of hyperparameters) for use in the simulation architecture 110. For instance, in the example embodiment, the simulation computing device 102 may consider hyperparameters such as batch size (e.g., how many data points architecture 110 sees each time (e.g., each epoch, etc.), how many data points the mode layer of the architecture 110 sees each time, how many data points each mode 112, 114 of the architecture 102 sees each time, etc.), a dimension of the latent genotype feature (e.g., the genotype mode 112, etc.) and/or a dimension of the latent environmental feature (e.g., the environment mode 114, etc.) in the latent space (e.g., in the mode layer, etc.), a learning rate (e.g., how much weights are updated after each batch, etc.), a number of layers in each module, and a given/particular deep learning algorithm used in each of the modes 112, 114, etc. In connection with the above, it should be appreciated that the computing device 102 further may consider, evaluate, etc. hundreds of thousands, millions, multiple millions, etc. of weights in the mode layer, may evaluate data points hundreds of times, thousands of times, etc. Further, in some examples, selections of the hyperparameter(s) for use in the architecture 110 (e.g., for use n subsequent simulation operations, etc.) may be evaluated and/or compared, relative to one another, based on one or more desired criteria such as, for example, mean absolute error (MEA), etc.

Then, once the desired hyperparameter(s) is/are identified for use in the architecture 110, one or more customized loss functions are defined to guide the model to converge on the training dataset. In the illustrated embodiment, and without limitation, the loss function(s) include three loss components. The first loss component (Loss 1) is the Cosine Similarity between the environmental latent vectors (e.g., of the environmental mode 114, etc.) and the genotype latent vectors (e.g., of the genotype mode 112, etc.), which serves to regularize during training. The range of this term is between −1 and 1. When it is a negative number between −1 and 0, 0 indicates orthogonality, while values closer to −1 indicate greater similarity. When the model converges during the training, the value will get close to −1, which indicates the environmental and genotype latent vectors are more aligned in the latent space, which may aid the simulation architecture 110 to learn the interaction effect of genotype and environment.

Loss 1=CosineSimilarity(x,y)

The second loss component (Loss 2) represents loss, where t is the trait of interest (or ear height in this example), i is non-missing data, y is the observed trait value in the training data (e.g., ear height (EHT), etc.), ŷ is the simulated trait value, and N is the number of uniquely indexable plots in the training data set. The second loss component serves as a regularizer to constrain the feature distribution in latent space to be close to the standard multivariate normal distribution. The effect of this second loss component is to normalize the latent features in the latent space.

$$Loss 2 = {\sum }_{t} \frac{1}{{N}_{t}} {\sum }_{i}^{{N}_{t}} ( {y}_{i}^{t} - {\hat{y}}_{i}^{t} )$$

The third loss component (Loss 3) measures the difference between a simulated trait value and an observed trait value, as part of a Kullback-Leibler (KL) divergence. The purpose of training process is to make the difference between these values as small as possible. This third loss component, then, guides the model to learn features related to the target trait from both genotype and environmental data. In the third loss component, then, the KL divergence is used to measure the difference between the multivariate normal distribution and the standard multivariate normal distribution. In connection therewith, N represents the number of uniquely indexable plots in the training data set, u represents a predicted mean trait, σ represents a precited standard deviation/scale of the trait, and I represents a unique plot index in the training set.

Loss 3=KL(N(μ,σ),(0,I))

The simulation computing device 102 is configured to rely on the training data set, and the loss function(s)/component(s) to then tune the hyperparameters of the simulation architecture.

It should be appreciated that the loss components may be used in combination or individually (or separately), whereby one or more of the loss components may be omitted from training of the architecture 110. In addition, it should be appreciated that the loss components described above may be used in connection with training the model for different traits (e.g., the loss components may not change in using the model to train other traits, etc.). That said, the trained model should only be used for simulation of a trait for which it is particularly trained (e.g., the model trained for simulating corn near height may not be available for use to simulate corn yield, etc.).

Once the simulation architecture 110 is trained, the simulation architecture 110 configures the simulation computing device 102 to simulate the ear height based on the above data.

As such, a grower may then select various different seed products to be planted in fields 104a-c, or one or more other fields. The grower may then identify the seed products to the simulation computing device 102.

In turn, the simulation computing device 102 is configured to access data from the database 106, which includes genotypic data for each of the potential seed products to be planted in the fields 104a-c and also weather and soil data for the fields 104a-c. The weather data may include a time series for one season, or an aggregate of weather data for multiple seasons (e.g., average temperature, per day, etc.). The soil data may likewise be associated with a current soil condition or historical soil conditions for the specific fields 104a-c. In general, the environmental data is compiled in a manner, in simulating the trait of interest, to approximate the environmental conditions of the specific field(s) 104a-c. As such, the environmental data may be compiled in a number of different manners.

In addition, the simulation computing device 102 is configured to access the trained simulation architecture 110. The simulation computing device 102 is then configured, by the simulation architecture 110, to simulate the trait of interest, or ear height in this example, for the selected/identified seed products in the specific fields 104a-c.

As noted above, management practices are also considered in the simulation architecture 110. Consequently, the management practice data may be modified or adjusted in different simulations to assess the seed products, alone or in combination with the management practices.

The simulation computing device 102 is configured to compare the simulated ear height of the different seed products to a threshold (e.g., about twenty inches, about twenty-four inches, about thirty inches, etc.) and to discard seed products which fail to satisfy the threshold. The simulation computing device 102 is configured to then present the remaining seed products (and management practices), if any, to the grower as options for planting in the fields 104a-c.

The grower then selects one or more of the remaining seed products, and an agricultural implement (e.g., a planter, etc.) is configured to plant the selected one or more seed products in the fields 104a-c. The agricultural implement (e.g., sprayer, etc.) may further impose the management practices, if any, during growing of the seed products in the fields 104a-c.

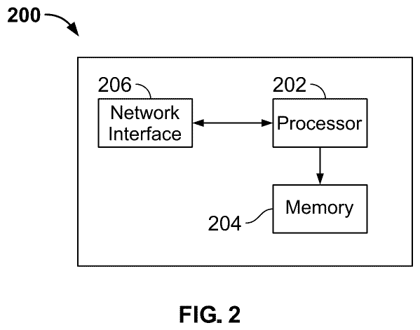

FIG. 2 illustrates an example computing device 200 that can be used in the system 100. The computing device 200 may include, for example, one or more servers, workstations, personal computers, laptops, tablets, smartphones, PDAs, virtual devices, etc. In addition, the computing device 200 may include a single computing device, or it may include multiple computing devices located in close proximity or distributed over a geographic region, so long as the computing devices are specifically configured to function as described herein. In the example embodiment of FIG. 1, the simulation computing device 102 and the database 106 are each implemented in and/or each include a computing device consistent with the computing device 200. With that said, the system 100 should not be considered to be limited to the computing device 200, as described below, as different computing devices and/or arrangements of computing devices may be used. In addition, different components and/or arrangements of components may be used in other computing devices.

Referring to FIG. 2, the example computing device 200 includes a processor 202 and a memory 204 coupled to the processor 202. The processor 202 may include one or more processing units (e.g., in a multi-core configuration, etc.). For example, the processor 202 may include, without limitation, one or more processing units such as a central processing unit (CPU), a microcontroller, a reduced instruction set computer (RISC) processor, an application specific integrated circuit (ASIC), a programmable logic device (PLD), a gate array, and/or any other circuit or processor capable of the functions described herein (alone or in combination).

The memory 204, as described herein, is one or more devices that permit data, instructions, etc., to be stored therein and retrieved therefrom. The memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media. The memory 204 may include one or more data structures and may be configured to store, without limitation, weather data, soil data, genotypic data, other data, trait data, and/or other types of data suitable for use as described herein.

Furthermore, in various embodiments, computer-executable instructions may be stored in the memory 204 for execution by the processor 202 to cause the processor 202 to perform one or more of the functions described herein (e.g., in the method 300, etc.), such that the memory 204 is a physical, tangible, and non-transitory computer readable storage media. Such instructions often improve the efficiencies and/or performance of the processor 202 that is operating as described herein (e.g., performing one or more of the operations of the method 300, etc.) whereby upon such performance of the one or more functions, the computing device 200 may be considered (or transformed into) a unique, special purpose device. It should be appreciated that the memory 204 may include a variety of different memories, each implemented in one or more of the functions or processes described herein.

In addition, the illustrated computing device 200 also includes a network interface 206 coupled to the processor 202 and the memory 204. The network interface 206 may include, without limitation, a wired network adapter, a wireless network adapter, a mobile network adapter (e.g., an NFC adapter, a Bluetooth adapter, etc.), or other device capable of communicating to one or more different networks. Further, in some example embodiments, the computing device 200 may include the processor 202 and one or more network interfaces incorporated into or with the processor 202.

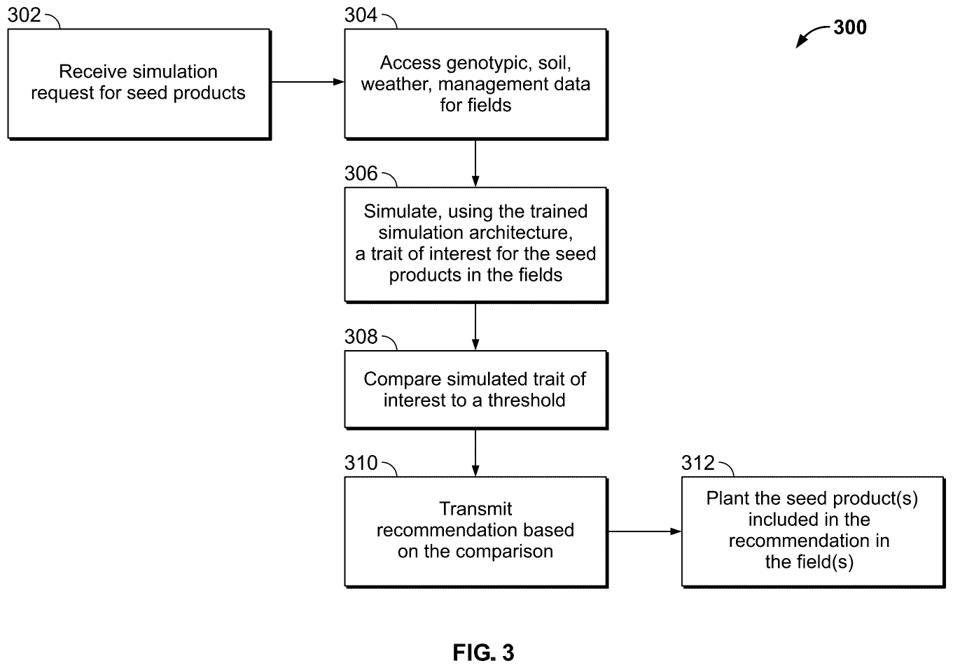

FIG. 3 illustrates an example method 300 for simulating one or more traits of interest based on genotypic data and environmental data. The example method 300 is described with reference to FIG. 1 as implemented in the simulation computing device 102 and as based on data associated with the fields 104a-c, etc., and also with reference to the computing device 200. However, it should be understood that the methods herein are not limited to the example system 100 or the example computing device 200. Likewise, the systems and the computing devices herein should not be understood to be limited to the example method 300.

At the outset in the method 300, it should be appreciated that one or more seed products are being evaluated for planting in one or more of the fields 104a-c. In connection therewith, a grower associated with the fields 104a-c, or a seed provider associated with the grower, defines the one or more seed products and also the fields 104a-c in which the one or more seed products are to be planted. The one or more seed products are defined by tradename, product number, or another identifier. In this example, the seed product type is short corn, and the seed product(s) include between five and twenty fives different varieties of short corn. Similarly, the fields 104a-c are identified by field ID, location, boundary, etc.

The seed products and fields 104a-c are compiled into a simulation request, which is submitted to the simulation computing device 102.

In turn, at 302, the simulation computing device 102 receives the simulation request for the seed products.

At 304, the simulation computing device 102 access data specific to the simulation request from the database 106. Initially, for example, the simulation computing device 102 searches for the seed products based on the identifier and accesses, based on the search, the genotypic data for each of the seed products. As explained above, the genotypic data includes vectors or matrixes indicative of a plurality of markers in the sequence of each variety with values indicative of the particular nucleotides at those markers.

Further, the simulation computing device 102 searches for the fields 104a-c identified in the simulation request, based on the associated identifiers, and accesses, based on the search, the weather data and soil data for the fields 104a-c. As explained above, the weather and soil data may be combined into a data structure, through normalization and/or resizing of the different resolutions of the data (e.g., in time and/or space, etc.). Additionally, the simulation computing device 102 access data associated with the management practices of the fields 104a-c, historically and also, potentially, as expected in the simulated growing season (e.g., expected or planned management practices, etc.).

In addition to the data, the simulation computing device 102 also accesses the trained simulation architecture 110. The simulation architecture 110 is trained based on a training data set consistent with the above accessed data, and also historical data for the trait of interest, etc., and ones of the loss functions also noted above. In this example embodiment, the trait of interest is corn ear height (EHT), as explained above. That said, in other embodiments, the trait of interest may be otherwise (e.g., yield, moisture, other traits described herein, etc.).

Next, at 306, the simulation computing device 102 simulates, using the trained simulation architecture 110, the trait of interest (e.g., ear height, etc.) for each of the fields 104a-c and each of the seed products. In this way, the simulation computing device 102 iteratively simulates ear height for the combinations of seed products and fields 104a-c, whereby each field is associated with each seed product and an ear height (e.g., in inches, etc.) for each seed product. As such, the simulation computing device 102 may provide multiple simulated trait values (e.g., ear height in this example, etc.) for the given seed products, for each of the given fields 104a-c. As an example, for one-hundred smart corn products and ten thousand potential field locations in which the smart corn products may be planted, the simulation computing device 102 may provide one million simulated values, with each value representing a simulated ear height of one of the smart corn products at one of the field locations.

In this example embodiment, the simulation computing device 102 then compares each of the simulated ear heights to a defined threshold, at 308. The defined threshold may include the operating height of agriculture implement (e.g., combine, etc.) located at one or more of the fields 104a-c. For example, the defined threshold may be twenty-four inches, as defined by an example combine. In another example, the defined threshold may include a buffer of two or more inches, whereby, for example, the defined threshold is twenty-eight inches (e.g., twenty-four inches plus a four inch buffer, etc.).

Based on the defined threshold, the simulation computing device 102 then transmits, at 310, one or more recommendations to the grower, or the seed provider, in response to the simulation request. The recommendation(s) may include one or more seed products, per field, along with the simulated ear height and/or an indication of the ear height relative to the defined threshold. It should be appreciated that the recommendation(s) may be provided in various forms. For example, the recommendation(s) may include a script for controlling an agricultural implement (e.g., a planter, etc.) to plant the recommended seed product(s) in the respective field(s). And, in turn, the seed products associated with the recommendation(s) are planted, at 312, in the field(s) 104a-c.

It should be appreciated from the above that the simulation architecture 110, in connection with system 100 and the method 300, provides precise and accurate simulation of different traits. In addition, it should be appreciated that the simulations of the different traits may be used in various different manners. For instance, the simulations may be used (as discussed above) in connection with making determinations of seeds to be planted in fields (e.g., recommendations of what seeds to plant in what fields, etc.) and/or whether the seeds should even be planted at all. The simulations may also be used in connection with testing and breeding decisions (e.g., which seeds to include in testing or in a breeding pipeline, etc.).

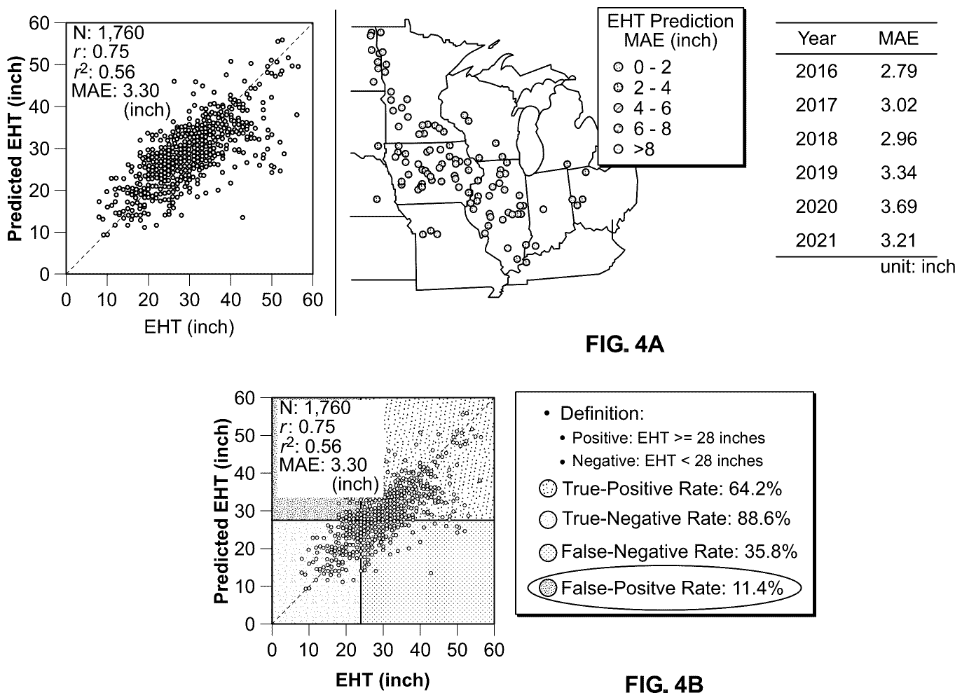

In connection with a simulation of thousands of data points over two, three, five, ten, etc. years, the simulation architecture 110 provides example performance as indicated in FIG. 4A. In this example, the time-series weather data used in ear height (EHT) simulation included 160 days of ERA5, TWC HoD, and TWC Hi-Res Precipitation data collected for each location from typical planting date for a span of six years (Year 1 to Year 6). All soil data was from the SoilGrid data source, and included the following features: OM, cec, ph, sand percentage, clay percentage, silt percentage, wwp, and bulk density. Genotypic data includes SNP marker datasets for all products. The typical planting date at county-level was aggregated and averaged and used to map cross-year planting dates to each corresponding data point based on geometry. A total of 130,000 data points were simulated for each product in each year. As shown, in a comparison of the simulated ear height (y-axis) relative to the measured ear height (x-axis), the diagonal line illustrates matching between the ear heights, whereby distance from the diagonal indicates deviation between the ear heights. A means absolute error (MAE) for the data indicates a deviation of about 3.3 inches over the ten years of data. Further data, which shows the MAE for discrete years of data, is also shown. Also, the map plot illustrates the limited number of data points which exceed 4 inch MAE.

Further, in FIG. 4B, a grid is overlaid on the plot from FIG. 4A. The grid is representative of the defined threshold, whereby a positive ear height is greater than or equal to twenty-eight inches, while a negative ear height is less than twenty-eight inches. Based on the simulated ear heights, relative to the measured ear eights, the performance of the simulation architecture 110 is shown in the rates of true-positive, true-negative, false-positives, and false negatives as shown.

In view of the above, the system and method provide for simulating traits associated with seed products in multiple fields based on data representative of the seed products and environment of the multiple fields.

In particular, in the embodiments herein, technology underlying the prediction/simulation of traits is improved, through inclusion of a multi-model model layer and an aggregation layer to permit specific processing, through the mode layer models, of widely disparate types of data (e.g., marker-based genetic data, time-based weather data, and depth-based soil data, etc.). In this manner, the disparate data is accurately and precisely relied on in predicting specific phenotypic traits (e.g. corn ear height, etc.), yet without forfeiting or diminishing impact of any one of the one or more data types to the other one or more data types, as with conventional single model, linear model, etc., approaches, where the data is combined before being introduced or input to the model. This serves to improve the underlying technology of the architecture herein and the resulting predictions/simulations therefrom.

With that said, it should be appreciated that the functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof (e.g., to adjust or adopt or scale yield data collected at pickers (broadly, harvesters or harvesting machines) to account for errors in calibration (where such adjustment may be performed or achieved at computing devices located away from the pickers, etc.), etc.), wherein the technical effect may be achieved by performing at least one (or more) of the following operations in the claims: (a) retrieving, from a data structure, data specific to multiple seed products and multiple fields, the data including a first mode of data and a second mode of data; (b) simulating a trait of interest for the multiple seed products and/or multiple fields, based on a simulation architecture, the simulation architecture including a first model specific to the first mode of data, a second model specific to the second mode of data, and a neural network coupled to the output of the first and second models as an aggregation layer; (c) transmitting the simulated trait of interest for the multiple seed products and/or multiple fields to a grower and/or an agricultural device associated with the multiple fields; (d) receiving a simulation request, the simulation request including identifiers for each of the multiple fields and/or the multiple seed products; and/or (e) causing/directing/instructing planting one or more of the multiple seed products included in the recommendation in one or more of the multiple fields.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for use in simulating one of more traits associated with seed products in multiple fields, the method comprising:
retrieving, by a simulation computing device, from a data structure, data specific to multiple seed products and multiple fields, the data including a first mode of data and a second mode of data;
simulating, by the simulation computing device, a trait of interest for the multiple seed products and/or multiple fields, based on a simulation architecture, the simulation architecture including a first model specific to the first mode of data, a second model specific to the second mode of data, and a neural network coupled to the output of the first and second models as an aggregation layer; and
transmitting the simulated trait of interest for the multiple seed products and/or multiple fields to a grower and/or an agricultural device associated with the multiple fields.

2. The computer-implemented method of claim 1, wherein the first mode of data includes genotypic data for the multiple seed products; and/or
wherein the second mode of data includes environmental data for the multiple fields; and
wherein the environmental data includes weather data and/or soil data.

3. The computer-implemented method of claim 1, wherein the trait of interest includes ear height; and
wherein the multiple seed products include multiple short corn seed products.

4. The computer-implemented method of claim 1, wherein the first model includes one or more of: a deep neural network (DNN) model, a convolutional neural network (CNN) model, a recurrent neural network (RNN) model, a generative pre-trained (GPT) model, a multilayer perceptron (MLP) model, and/or a long short-term memory (LSTM) model; and/or
wherein the second model includes one or more of: a DNN model, CNN model, a RNN model, a GPT model, a MLP model, and/or a LSTM model; and/or
wherein the neural network includes a feed-forward network, a multi-head neural network, and/or a feed forward network with residual connections.

5. The computer-implemented method of claim 1, further comprising receiving a simulation request, the simulation request including identifiers for each of the multiple fields and/or the multiple seed products; and
wherein retrieving the data includes searching for the identifiers for each of the multiple fields and/or the multiple seed products.

6. The computer-implemented method of claim 1, wherein the output of the first and second models includes latent feature data, which is combined prior to input to the aggregation layer.

7. The computer-implemented method of claim 1, wherein the aggregation layer includes a neural network model.

8. The computer-implemented method of claim 1, further comprising planting one or more of the multiple seed products included in the recommendation in one or more of the multiple fields.

9. A system for use in simulating one of more traits associated with seed products in multiple fields, the system comprising at least on computing device configured to:
retrieve, from a data structure, data specific to multiple seed products and multiple fields, the data including a first mode of data and a second mode of data;
simulate a trait of interest for the multiple seed products and/or multiple fields, based on a simulation architecture, the simulation architecture including a first model specific to the first mode of data, a second model specific to the second mode of data, and a neural network coupled to the output of the first and second models as an aggregation layer; and
transmit the simulated trait of interest for the multiple seed products and/or multiple fields to a grower and/or an agricultural device associated with the multiple fields.

10. The system of claim 9, wherein the first mode of data includes genotypic data for the multiple seed products; and/or
wherein the second mode of data includes environmental data for the multiple fields; and
wherein the environmental data includes weather data and/or soil data.

11. The system of claim 10, wherein the trait of interest includes ear height; and
wherein the multiple seed products include multiple short corn seed products.

12. The system of claim 11, wherein the first model includes one or more of: a deep neural network (DNN) model, a convolutional neural network (CNN) model, a recurrent neural network (RNN) model, a generative pre-trained (GPT) model, a multilayer perceptron (MLP) model, and/or a long short-term memory (LSTM) model; and/or
wherein the second model includes one or more of: a DNN model, CNN model, a RNN model, a GPT model, a MLP model, and/or a LSTM model; and/or
wherein the neural network includes a feed-forward network, a multi-head neural network, and/or a feed forward network with residual connections.

13. The system of claim 9, wherein the at least one computing device is further configured to receive a simulation request, the simulation request including identifiers for each of the multiple fields and/or the multiple seed products; and
wherein the at least one computing device is configured, in order to retrieve the data, to search for the identifiers for each of the multiple fields and/or the multiple seed products.

14. The system of claim 9, wherein the output of the first and second models includes latent feature data, which is combined prior to input to the aggregation layer.

15. The system of claim 14, wherein the aggregation layer includes a neural network model.

16. The system of claim 9, wherein the at least one computing device is configured to transmit instructions to at least one agricultural implement to cause the at least one agricultural implement to plant one or more of the multiple seed products included in the recommendation in one or more of the multiple fields.

17. A non-transitory computer readable storage medium including executable instructions, which, when executed by at least one processor for simulating one of more traits associated with seed products in multiple fields, cause the at least one processor to:
retrieve, from a data structure, data specific to multiple seed products and multiple fields, the data including a first mode of data and a second mode of data;
simulate a trait of interest for the multiple seed products and/or multiple fields, based on a simulation architecture, the simulation architecture including a first model specific to the first mode of data, a second model specific to the second mode of data, and a neural network coupled to the output of the first and second models as an aggregation layer; and
transmit the simulated trait of interest for the multiple seed products and/or multiple fields to a grower and/or an agricultural device associated with the multiple fields.

18. The non-transitory computer readable storage medium of claim 17, wherein the first mode of data includes genotypic data for the multiple seed products; and wherein the second mode of data includes environmental data for the multiple fields; and wherein the environmental data includes weather data and/or soil data; and
wherein the trait of interest includes ear height; and wherein the multiple seed products include multiple short corn seed products.

19. The non-transitory computer readable storage medium of claim 18, wherein the first model includes one or more of: a deep neural network (DNN) model, a convolutional neural network (CNN) model, a recurrent neural network (RNN) model, a generative pre-trained (GPT) model, a multilayer perceptron (MLP) model, and/or a long short-term memory (LSTM) model;
wherein the second model includes one or more of: a DNN model, CNN model, a RNN model, a GPT model, a MLP model, and/or a LSTM model;
wherein the neural network includes a feed-forward network, a multi-head neural network, and/or a feed forward network with residual connections.

20. The non-transitory computer readable storage medium of claim 19, wherein the executable instructions, when executed by the at least one processor, further cause the at least one processor to receive a simulation request, the simulation request including identifiers for each of the multiple fields and/or the multiple seed products; and
wherein the executable instructions, when executed by the at least one processor to retrieve the data, cause the at least one processor to search for the identifiers for each of the multiple fields and/or the multiple seed products.
